(19) United States
(12) Patent Application Publication
(10) Pub. No.: US 2022/0348999 A1
(43) Pub. Date: Nov. 3, 2022

(54) Normalization of Nucleic Acid Samples and Compositions for Use in the Same

(71) Applicant: Takara Bio USA, Inc., Mountain View, CA (US)

(72) Inventors: Kazuo Tori, Mountain View, CA (US); Andrew Alan Farmer, Mountain View, CA (US); Yoshitaka Kimura, Ann Arbor, MI (US)

(21) Appl. No.: 17/277,230

(22) PCT Filed: Dec. 4, 2019
(86) PCT No.: PCT/US2019/064477

§ 371 Date: Mar. 17, 2021

Related U.S. Application Data
(60) Provisional application No. 62/781,228, filed on Dec. 18, 2018.

(51) Int. Cl. C12Q 1/6869; C12N 15/10; C12N 9/22; C12N 15/11

(52) U.S. Cl. CPC C12Q 1/6869; C12N 15/1065; C12N 9/22; C12N 15/11; C12N 2800/80; C12N 2310/20

(57) ABSTRACT

Methods of normalizing two or more nucleic acid samples are provided. Aspects of the methods include contacting each of the two or more nucleic acid samples with a limiting amount of a target binding moiety that specifically binds to a common target in each of the two or more nucleic acid samples to produce binding complexes in each of the two or more nucleic acid samples; and separating the binding complexes from unbound nucleic acids in each of the two or more nucleic acid samples to normalize the two or more nucleic acid samples. Compositions and kits for use in performing the methods are also provided.

### CROSS-REFERENCE TO RELATED APPLICATIONS

Pursuant to 35 U.S.C. § 119(e), this application claims priority to the filing date of the U.S. Provisional Patent Application Ser. No. 62/781,228 filed Dec. 18, 2018; the disclosure of which application is herein incorporated by reference.

### INTRODUCTION

The development of next generation sequencing (NGS) technologies has allowed for the rapid extraction of valuable genomic and transcriptomic information from produced nucleic acid libraries. High throughput NGS technologies, such as Illumina (Solexa) sequencing, Roche 454 sequencing, Ion torrent (Proton/PGM sequencing) and SOLiD sequencing, allow the sequencing of nucleic acid molecules more quickly and cheaply than previously used Sanger sequencing, and as such these techniques have revolutionized biotechnology and biomedical research. These powerful sequencing technologies place a particular emphasis on library preparation.

In NGS protocols, multiple library preparations, e.g., prepared from single cells, are often multiplexed, e.g., by pooling, prior to sequencing. Pooling multiple library preparations to prepare multiplexed libraries for subsequent sequencing can provide a number of advantages, including maximization of NGS technology capacity utilization, reduction of reagent use, etc.

Where multiplexed libraries are employed in NGS protocols, it is often desirable to normalize the different nucleic acids libraries that are pooled. Normalization may be viewed as the process of equalizing the DNA library concentration for multiplexing and addresses the problems of library over-representation or under-representation in a given multiplexed composition. In a given multiplex NGS workflow, normalization may be employed at different stages, including normalization of the concentration of input DNA/RNA, size distribution of library fragments as well as the normalization of library preparation concentration prior to pooling.

Many NGS protocols include quantitatively checking individual library preparations followed by adjustment of the libraries to equimolar ratios before pooling. In such instances, a number of different approaches may be employed, including spectrophotometry, electrophoresis, fluorometry, quantitative PCR (qPCR), and magnetic bead normalization. A continued need exists for the development of alternative normalization protocols.

### SUMMARY

Methods of normalizing two or more nucleic acid samples are provided. Aspects of the methods include contacting each of the two or more nucleic acid samples with a limiting amount of a target binding moiety that specifically binds to a common target in each of the two or more nucleic acid samples to produce binding complexes in each of the two or more nucleic acid samples; and separating the binding complexes from unbound nucleic acids in each of the two or more nucleic acid samples to normalize the two or more nucleic acid samples. Compositions and kits for use in performing the methods are also provided.

### BRIEF DESCRIPTION OF THE FIGURES

FIG. 1A provides a schematic representation of a protocol for normalizing two dsDNA libraries according to an embodiment of the invention.

FIG. 1B provides a schematic representation of a protocol for normalizing two dsDNA libraries according to an embodiment of the invention.

FIG. 2 provides a schematic representation of a protocol for normalizing two dsDNA libraries according to another embodiment of the invention.

FIG. 3 provides a schematic representation of a protocol for normalizing two dsDNA libraries according to another embodiment of the invention.

FIG. 4 provides a schematic representation of a protocol for reducing the amounts of primer-dimers from dsDNA libraries according to an embodiment of the invention.

### DEFINITIONS

As used herein, the term “hybridization conditions” means conditions in which a primer, or other polynucleotide, specifically hybridizes to a region of a target nucleic acid with which the primer or other polynucleotide shares some complementarity. Whether a primer specifically hybridizes to a target nucleic acid is determined by such factors as the degree of complementarity between the polymer and the target nucleic acid and the temperature at which the hybridization occurs, which may be informed by the melting temperature (T_{M}) of the primer. The melting temperature refers to the temperature at which half of the primer-target nucleic acid duplexes remain hybridized and half of the duplexes dissociate into single strands. The Tm of a duplex may be experimentally determined or predicted using the following formula Tm=81.5+16.6(log 10[Na+])+0.41 (fraction G+C)−(60/N), where N is the chain length and [Na+] is less than 1 M. See Sambrook and Russell (2001; Molecular Cloning: A Laboratory Manual, 3rd ed., Cold Spring Harbor Press, Cold Spring Harbor N.Y., Ch. 10). Other more advanced models that depend on various parameters may also be used to predict Tm of primer/target duplexes depending on various hybridization conditions. Approaches for achieving specific nucleic acid hybridization may be found in, e.g., Tijssen, Laboratory Techniques in Biochemistry and Molecular

Biology-Hybridization with Nucleic Acid Probes, part I, chapter 2, “Overview of principles of hybridization and the strategy of nucleic acid probe assays,” Elsevier (1993).

The terms “complementary” and “complementarity” as used herein refer to a nucleotide sequence that base-pairs by non-covalent bonds to all or a region of a target nucleic acid (e.g., a region of the product nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotides are complementary to every nucleotide in the target nucleic acid in all the corresponding positions. For example, a primer may be perfectly (i.e., 100%) complementary to the target nucleic acid, or the primer and the target nucleic acid may share some degree of complementarity which is less than perfect (e.g., 70%, 75%, 85%, 90%, 95%, 99%).

The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., % identity=# of identical positions/total # of positions×100). When a position in one sequence is occupied by the same nucleotide as the corresponding position in the other sequence, then the molecules are identical at that position. A non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

### DETAILED DESCRIPTION

Methods of normalizing two or more nucleic acid samples are provided. Aspects of the methods include contacting each of the two or more nucleic acid samples with a limiting amount of a target binding moiety that specifically binds to a common target in each of the two or more nucleic acid samples to produce binding complexes in each of the two or more nucleic acid samples; and separating the binding complexes from unbound nucleic acids in each of the two or more nucleic acid samples to normalize the two or more nucleic acid samples. Compositions and kits for use in performing the methods are also provided.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods

As reviewed above, methods of normalizing two or more nucleic acid samples are provided. By “normalizing” is mean that the nucleic acid concentration among two or more nucleic acid samples is evened out or made at least substantially equal, if not identical, e.g., by adjusting the nucleic acid concentration in the libraries to equimolar ratios. As such, among nucleic acid samples normalized by methods of the invention, the nucleic acid concentration is substantially the same, such that any variation in concentration, if present, is minimal. If determined using a spectrophotometric protocol, the magnitude of any nucleic acid concentration variation among normalized samples produced in accordance with embodiments of the invention may, is some instances, be five-fold or less, such as three-fold or less, including 0.1-fold or less. Methods of the invention may be employed to normalize a plurality of nucleic acid samples, where the term plurality refers to two or more, such as three or more, four or more, including five or more, e.g., ten or more, twenty or more, 100 or more, 1,000 or more, 10,000 or more, etc., where in some instances the number of distinct nucleic acid samples that are normalized ranges from two to 20,000, such as two to 10,000.

Nucleic acid samples that may be normalized in accordance with embodiments of the invention may vary, where in some instances the nucleic acid samples are compositions made up of a plurality of distinct nucleic acids (e.g., corresponding to distinct genes) that differ from each other in terms of overall sequence. While the number of distinct nucleic acids in a given nucleic acid sample may vary, in some instances the number of distinct nucleic acids present in a given nucleic acid sample is 10 or more, such as 25 or more, 50 or more, 100 or more, 500 or more, 1000 or more, 10,000 or more, 20,000 or more, where in some instances the number of distinct nucleic acids ranges from 1,000 to 25,000, such as 2,000 to 20,000. The nucleic acid constituents of a given nucleic acid sample may be single stranded nucleic acids or double stranded nucleic acids, where in some instances the nucleic acids are double stranded deoxyribonucleic acids (dsDNAs). A variety of different types of nucleic acid samples may be normalized according to embodiments of the inventions, where examples of nucleic acid samples that may be normalized include, but are not limited to: next generation sequencing (NGS) libraries, microarray libraries, etc. Further details regarding types of libraries that may be normalized in accordance with methods of the invention, including the preparation thereof, are provided below.

In practicing embodiments of the invention, two or more nucleic acid samples, e.g., as reviewed above, are each contacted with the same limiting amount of a target binding moiety that specifically binds to a common target in nucleic acids of each of the two or more nucleic acid samples to produce binding complexes in each of the two or more nucleic acid samples. Following binding complex production, the resultant binding complexes are separated from unbound nucleic acids in each of the two or more nucleic acids to produce normalized nucleic acid samples from the two or more nucleic acid samples, i.e., to normalize the two or more nucleic acid samples.

### Normalization Binding Moieties

As summarized above, an aspect of the methods includes contacting two or more nucleic acid samples with a limiting amount of a normalization binding moiety. As used herein, a “normalization binding moiety” refers to an entity that specifically binds to a common target found in distinct nucleic acids that are to be normalized in the nucleic acid samples. The phrase “specifically binds” refers to the interaction of a pair of molecules that have binding specificity for one another, such that they preferentially bind to each other as opposed to other molecules that may be present in theft environment. As the normalization binding moiety specifically binds to the common target, it preferentially binds to the common target as opposed to other entities, e.g., other nucleic acid sequences, that may be present in the nucleic acid samples.

In embodiments of the invention, the normalization binding moiety is a solution phase entity, i.e., it is not a solid phase entity, such as a bead or particle, e.g., a magnetic bead. In yet other instances, the normalization binding moiety may be stably associated with, e.g., covalently or non-covalently bound to, the surface of a solid support, such as a bead, column component, membrane (e.g., Capturem™ high-capacity membranes, Takara Bio USA, Mountain View, Calif.), etc. In specific embodiments where the normalization binding moiety is a stably associated with a solid phase, it is not a non-sequence specific binding moiety, e.g., avidin/streptavidin or non-specific DNA binding protein, a polyT binding moiety, chemical group, etc.

In some instances, the normalization binding moiety comprises a biomolecule, such as a nucleic acid, polypeptide, lipid, etc. Of interest in certain embodiments are proteinaceous normalization binding moieties, i.e., moieties that include a protein component, where such normalization binding moieties may be referred to as normalization binding proteins. Examples of proteinaceous normalization binding moieties include normalization binding moieties that include a normalization binding protein capable of specifically binding to a common target that is present in nucleic acids of the nucleic acid samples, e.g., libraries, that are to be normalized (e.g., in order to allow for normalization methods of the disclosure to occur). Normalization binding proteins employed in embodiments of the invention may vary. A normalization binding protein can bind to any type of common target that may be present in the nucleic acid constituents of the two or more nucleic acid samples, where common targets of interest include, but are not limited to, nucleic acid sequences, nucleic acid secondary structures, e.g., (e.g., hairpins, termini, etc.), non-nucleic acid tags associated with the nucleic acids, etc. Normalization binding proteins that may be employed include, but are not limited to: single stranded binding proteins (SSBs), transposases, recombinases, methylases, histones, Sul7D family of archaeal chromatin proteins, among others, e.g., as further reviewed below.

In some instances, the normalization binding protein is a nucleic acid binding protein, e.g., a DNA binding protein, that specifically binds to a target moiety. In some such instances, the nucleic acid binding protein is a sequence specific normalization binding protein, by which is meant that the normalization binding protein specifically binds to a common target nucleic acid sequence that is present in the constituent nucleic acid members that are to be normalized in the nucleic acid samples. Examples of such normalization binding proteins include, but are not limited to: nucleases, transcription factors, and the like.

In some instances, the normalization binding protein is a nuclease. The nuclease may be catalytically active or inactive, as desired, where in some instances the nuclease is catalytically inactive. Examples of nucleases of interest include, but are not limited to, nucleic add guided endonucleases, restriction endonucleases, etc. As used herein, a “nucleic acid guided endonuclease” is an association (e.g., a complex) that includes a nuclease component and a nucleic acid guide component. The nucleic acid guided endonuclease may be catalytically inactive, where the endonuclease is a modified nuclease that does not have nuclease activity (e.g., is cleavage deficient) as a result of the modification. A catalytically inactive endonuclease is a mutant that is cleavage deficient—e.g., Sp, a Cas9 D10A mutant, a Cas9 H840A mutant, a Cas9 D10A/H840A mutant, or any other suitable cleavage deficient mutant. Endonuclease domains from which a catalytically nuclease/cleavage deficient domain can be derived include, but are not limited to: a Cas nuclease (e.g., a Cas9 nuclease), an Argonaute nuclease (e.g., Tth Ago, mammalian Ago2, etc.), S1 Nuclease; rung bean nuclease; pancreatic DNase I; micrococcal nuclease; yeast HO endonuclease; a restriction endonuclease; a homing endonuclease; and the like; see also Mishra (Nucleases: Moleccular Biology and Applications (2002) ISBN-10: 0471394610).

As described above, according to certain embodiments, the nucleic add guided nuclease includes a CRISPR-associated (or “Cas”) nuclease (e.g., or catalytically inactive mutant thereof). The CRISPR/Cas system is an RNA-mediated genome defense pathway in archaea and many bacteria having similarities to the eukaryotic RNA interference (RNAi) pathway. The pathway arises from two evolutionarily (and often physically) linked gene loci: the CRISPR (clustered regularly interspaced short palindromic repeats) locus, which encodes RNA components of the system; and the Gas (CRISPR-associated) locus, which encodes proteins. Non-limiting examples of Cas proteins include Cast Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1 Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain aspects, the nuclease component of the nucleic acid guided nuclease is Cas9. The Cas9 may be from any organism of interest, including but not limited to, Streptococcus pyogenes (“spCas9”, Uniprot Q99ZW2) having a PAM sequence of NGG; Neisseria meningitidis (“nmCas9”, Uniprot C6S593) having a PAM sequence of NNNNGATT; Streptococcus thermophilus (“stCas9”, Uniprot Q5M542) having a PAM sequence of NNAGAA, and Treponerna denticois (“tdCas9”, Uniprot M2B9U0) having a PAM sequence of NAAAAC.

In certain aspects, the nuclease component of the nucleic acid guided nuclease is an Argonaute (ago) nuclease. Ago proteins are a family of evolutionarily conserved proteins central to the RNA interference (RNAi) platform and microRNA (miRNA) function and biogenesis. They are best known as core components of the RNA-induced silencing complex (RISC) required for small RNA-mediated gene regulatory mechanisms. In post-transcriptional gene silencing, Ago guided by a small RNA (e.g., sRNA, miRNA, piRNA, etc.) binds to the complementary transcripts via base-pairing and serve as platforms for recruiting proteins to facilitate gene silencing. Mammals have eight Argonaute proteins, which are divided into two subfamilies: the Piwi clade and the Ago clade. Of the wild-type Ago proteins (Ago1-4, or EIF2C1-4), only Ago2 has endonuclease activity. The crystal structure of full-length human Ago2 (Uniprot Q9UKV8) has been solved. Similar to the bacteria counterpart, human Ago2 is a bilobular structure comprising the N-terminal (N), PAZ, MID, and PIWI domains. The PAZ domain anchors the 3′ end of the small RNAs and is dispensable for the catalytic activity of Ago2. However, PAZ domain deletion disrupts the ability of the non-catalytic Agos to unwind small RNA duplex and to form functional RISC. When the nuclease component of the nucleic acid guided nuclease is an Ago nuclease, the nuclease may be an Ago nuclease that cleaves DNA duplexes, RNA duplexes, or DNA-RNA duplexes. The Ago nuclease may be derived from any suitable organism, such as a prokaryotic or eukaryotic organism. In certain aspects, the Ago is a prokaryotic Ago. Prokaryotic Agos of interest include, but are not limited to, Thermus thermophiles Ago (“Tth Ago”), such as the Tth Ago nucleases described in Wang et al. (2008) Nature 456(7224):921-926; and Wang et al. (2009) Nature 461(7265):754-761. DNA-guided DNA interference in vivo using Tth Ago and 5′-phosphorylated DNA guides of from 13-25 nucleotides in length was recently described by Swarts et al. (2014) Nature 507:258-261.

When the normalization binding protein comprises a nucleic acid guided nuclease (or catalytically inactive variant thereof), the nucleic acid guided nuclease includes a nucleic acid guide component. The nucleic acid guide component may be one or more nucleic acid polymers of any suitable length. In certain aspects, the nucleic acid guide component is a nucleic acid polymer (e.g., a single- or double-stranded RNA or DNA) of from 10 to 200 nucleotides in length, such as from 10 to 150 nucleotides in length, including from 10 to 100, from 10 to 90, from 10 to 80, from 10 to 70, from 10 to 60, from 10 to 50, from 10 to 40, from 10 to 30, from 10 to 25, from 10 to 20, or from 10 to 15 nucleotides in length. At least a portion of the nucleic acid guide component is complementary (e.g., 100% complementary or less than 100% complementary) to at least a portion of a target nucleic acid of interest. The sequence of all or a portion of the nucleic acid guide component may be selected by a practitioner of the subject methods to be sufficiently complementary to a target nucleic acid of interest to specifically guide the nuclease component to the target nucleic acid. The nucleic acid sequences of target nucleic acids of interest are readily available from resources such as the nucleic acid sequence databases of the National Center for Biotechnology Information (NCBI), the European Molecular Biology Laboratory-European Bioinformatics Institute (EMBL-EBI), and the like. According to certain embodiments, the nucleic acid guide component is an RNA guide component (or “guide RNA”). The RNA guide component may include one or more RNA molecules. For example, the RNA guide component may include two separately transcribed RNAs (e.g., a crRNA and a tracrRNA) which form a duplex that guides the nuclease component (e.g., Cas9) to the target nucleic acid. In other aspects, the RNA guide component is a single RNA molecule, or alternatively, may be an engineered single guide RNA. According to certain embodiments, the nucleic acid guide component is an engineered single guide RNA that includes a crRNA portion fused to a tracrRNA portion, which single guide RNA is capable of guiding a nuclease (e.g., Cas9) to the target nucleic acid. In certain aspects, the nucleic acid guide component is a DNA guide component, e.g., a single-stranded or double-stranded guide DNA. According to certain embodiments, the guide DNA is phosphorylated at one or both ends. For example, the guide DNA may be a 5′-phosphorylated guide DNA oligonucleotide of any suitable length (e.g., any of the lengths set forth above, including for example, from 10 to 30 nucleotides in length). As summarized above, embodiments of the methods of the present disclosure include contacting an initial collection of nucleic acids with a normalization binding protein (e.g., in some instances comprising a nucleic acid guided nuclease specific for the target nucleic acid of interest) in a manner sufficient to normalize the library (see disclosure below). In certain aspects, contacting the libraries with a normalization binding protein can include combining in a reaction mixture the library, a nucleic acid guide component, and a nuclease component. The nucleic acid guide component and the nuclease component may be stably associated (e.g., as a complex) prior to being added to the reaction mixture, or these components may be added separately for subsequent association with each other and targeting/depletion of the target nucleic acid.

Also of interest are restriction endonucleases. Restriction endonucleases are present in many species and are capable of sequence-specific binding to DNA (at a recognition site), and cleaving DNA at or near the site of binding. Certain restriction enzymes (e.g., Type IIs) cleave DNA at sites removed from the recognition site and have separable binding and cleavage domains. For example, the Type IIs enzyme FokI catalyzes double-stranded cleavage of DNA, at 9 nucleotides from its recognition site on one strand and 13 nucleotides from its recognition site on the other. Examples of the Type IIs restriction enzymes include FokI, AarI, AceIII, AciI, AloI, BaeI, Bbr7I, CdiI, CjePI, EciI, Esp3I, FinI, MboI, SapI, and SspD51, but are not limited thereto.

In some instances, the normalizing binding protein comprises a TAL effector domain, e.g., as found in transcription activator-like effector (TALEN) nucleases. As used herein, the term “transcription activator-like effector nuclease (TALEN)” refers to a class of highly specific restriction endonucleases that can be engineered to cut specific sequences of DNA and may include all known or commercial transcription activator-like effector nucleases. TALENs are fusion proteins comprising a TAL effector (TALE) DNA binding domain and a nucleotide cleavage domain. The TAL effector domain harbor highly conserved repeat domains that each bind to a single base pair of DNA. The identities of two residues (referred to as repeat variable di-residues or RVDs) in these 33 to 35 amino acid repeats are associated with the binding specificity of these domains. TAL effector repeats can be joined together to highly sequence specific restriction enzymes, which are capable of binding and cleaving target DNA sequences of interest.

In some instances, normalization binding proteins include a zinc finger domain. As used herein, the term “zinc finger domain” refers to a protein that binds to a nucleotide in a sequence-specific manner through one or more zinc finger modules. The zinc finger domain includes at least two zinc finger modules. The zinc finger domain is often abbreviated as zinc finger protein or ZFP. As used herein the term “zinc finger protein (ZFP)” refers to a polypeptide having nucleic acid (e.g., DNA) binding domains that are stabilized by zinc. The individual DNA binding domains are typically referred to as “fingers,” such that a zinc finger protein or polypeptide has at least one finger, more typically two fingers, or three fingers, or even four or five fingers, to at least six or more fingers. Each finger typically binds from two to four base pairs of DNA. Each finger usually comprises an about 30 amino acids zinc-chelating, DNA-binding region.

In yet other instances, the normalization binding protein comprises a transcription factor or a DNA-binding domain (DBD) thereof. Examples of DBDs that may be present in the normalization binding protein include, but are not limited to: basic helix-loop-helix DBDs, basic-leucine zipper DBDs, C-terminal effector domain of the bipartite response regulators DBDs, AP2/ERF/GCC box DBDs, helix-turn-helix DBDs, homeodomain DBDs, lambda repressor like DBDs, srf-like DBDs, paired box DBDs, winged helix DBDs, and zinc finger DBDs.

In yet other instances, the normalization binding protein specifically binds to a nucleic acid structural motif, e.g., a terminal end, a hairpin, etc. Examples of such normalization binding proteins include, but are not limited to: Ku, DNA-PK, TERF1, stem-loop binding protein (SLBP), and the like.

In yet other instances, the normalization binding protein specifically binds to a non-nucleic tag that is part of the nucleic acids to be normalized, which has been incorporated into the nucleic acids of the sample during preparation thereof, e.g., as a non-templated sequence, such as described below, e.g., arising from a primer, template switch oligonucleotide, adapter component, etc. In such instances, the normalization binding protein may bind to any of a variety of different types of tags, where tags of interest include, but are not limited to: Biotin, Digoxigenin, FITC, methylation, etc., which may be introduced into nucleic acid constituents of the samples by primers that include such tags. As such, normalization binding proteins in such embodiments may be streptavidin, specific binding members, e.g., antibodies or binding fragments thereof, methylation binding proteins, etc.

Where desired, the normalization binding moiety may include a purification domain. A purification domain is a region or portion of the normalization binding moiety that may be employed to separate binding complexes from other constituents, e.g., unbound nucleic acids, e.g., as described in greater detail below, e.g., to facilitate separation of the normalization binding protein bound to certain library molecules (e.g., by affinity purification) from the other components of the libraries being normalized. Any convenient purification domain may be employed, where examples of purification domains include, but are not limited to, tags, such as epitope tags, e.g., FLAG tag (DYKDDDDK, e.g., for purification via M1, M2, M5), HA tag (YPYDVPDYA, e.g., for purification via 120A5), His tag (e.g., 6xHis, HHHHHH, e.g., for purification via anti-His), Myc tag (EQKLISEEDL, e.g., for purification via 9E10), CD tag (18 aa exon, e.g., for purification via 12CA5), S-tag (S-peptide, e.g., for purification via anti-S peptide), SBP tag, Softag, GST tag (220 aa GST, e.g., for purification via anti-GST), GFP tag, Sumo tag, SNAP tag, strep tag (WSAPQFEK, e.g., for purification via Strep-Tactin), MBP tag (maltose-binding protein, e.g., for purification via anti-MBP), CBD tag (chitin-binding domain, e.g., for purification via anti-CBD), avitag (GLNDIFEAQKIEWHE, e.g., for purification via avidin), CBP tag (calmodulin binding protein peptide, e.g., for purification via anti-CBP), TAP tag (calmodulin and IgG-binding domains, e.g., for purification via anti-CBP), SF-TAP tag (Strep Tag II and FLAG, e.g., for purification via anti-Flag, biotin, streptavidin, covalent linkage tags, e.g., halo tags, glycopeptide tag (e.g., for binding to lectins, FC tag (e.g., for binding to Protein A or G), etc.

As reviewed above, in some instances the normalization binding moiety may be associated with the surface of a solid phase, e.g., a bead, column component, etc., such that the solid phase normalization binding moiety. For example, in embodiments where the normalization binding moiety comprises nucleic acid guided inactive nuclease, e.g., Cas, Ago, etc., beads may first prepared that include the sgRNA attached to them, e.g., using convenient solid phase synthesis protocols. Next, the sgRNA displaying bead may be combined with the inactive nuclease to produce a solid phase nucleic acid guided nuclease with is then used as a normalization binding protein in accordance with methods of the invention. In such instances, the guide oligo is easy to synthesize on the beads in defined amounts—ensuring that the amount is limiting with respect to the library, e.g., as described in greater detail below.

### Normalization Methods

As reviewed above, aspects of the methods include normalizing two or more, e.g., ten or more, twenty or more, 100 or more, 1,000 or more, 10,000 or more, etc., nucleic acid samples, e.g., libraries, using a normalization binding moiety, e.g., normalization binding protein, such as described above. In practicing embodiments of the methods, the normalization binding moiety is contacted with the nucleic acid samples to be normalized in a limiting amount. As used herein the phrase “limiting amount” refers to a concentration of a normalization binding moiety (e.g., of normalization binding protein) that is less than the nucleic acids having a common target that is bound by the normalization binding moiety. As such, when a limiting amount of normalization binding moiety is contacted to nucleic acids of the to be normalized nucleic acid samples, substantially all of the normalization binding moiety will end up being bound to nucleic acids in binding complexes, such that 80% or more, 90% or more, 95% or more, including 100% of the normalization binding moiety will end up in binding complexes with nucleic acids of the sample. Accordingly, the nucleic acids of a given sample that include a common target to which the normalization binding moiety specifically binds are in excess relative to the limiting amount of the normalization binding moiety, such that binding to the normalization binding moiety essentially goes to completion/saturation ensuring that the amount of nucleic acid sample, e.g., library, present at the end after normalization is quantitatively the same as the amount of normalization complex, thus normalizing the nucleic acid samples, e.g., libraries, all to the same defined fixed and limiting amount of the normalization binding moiety. The same limiting amount of normalization binding moiety is contacted with each of the nucleic acid samples, e.g., libraries, that are to be normalized.

As summarized above, constituent nucleic acids of the samples that are normalized in accordance with embodiments of the invention include a common target to which the normalization binding moiety specifically binds. As reviewed above, the common target may vary widely depending on the normalization binding moiety that is employed. Examples of common targets include, but are not limited to, nucleic acid sequences, structures or motifs, or non-nucleic acid tags, e.g., as described above. While the common target may be located in any convenient region of the constituent nucleic acids of the sample, in some instances the common target is near or at an end location of the nucleic acids, e.g., near or at a terminus of the nucleic acids such as within 100 bases or closer to the terminus, e.g., 50 bases or closer to the terminus, including 25 bases or closer to the terminus. The common target may be at target that is present in templated or non-templated regions of nucleic acids, e.g., as described in greater detail below. In some instances, the common target is present in a common adapter (e.g., sequencing adapter) of the nucleic acids of the sample. Libraries normalized by the methods of the disclosure can include common end sequences. For example, libraries that undergo tagmentation (e.g., Nextera tagmentation) can include a sequencing platform adaptor construct on each end (e.g., a P5 or P7 sequence on each end). Libraries can include multiple common sequences on each end. In some instance libraries can include one, two, three or more common sequences on one or more ends. In some instances, libraries include one common sequence on each end (e.g., P5 or P7 for sequencing). A normalization binding moiety of the disclosure can be designed to bind to one of the common end sequences of the libraries. Normalization binding proteins can be added to the libraries in a limiting amount such that the normalization binding protein is saturated with common end sequence containing library molecules.

In practicing embodiments of the methods, the same limiting amount of normalization binding moiety is contacted with each of the two or more nucleic acid samples that are to be normalized under conditions sufficient to produce binding complexes in each of the samples between normalization binding moieties and nucleic acids that include a common target to which the normalization binding moieties specifically bind. While the limiting amount may vary in a given protocol, in some instances the limiting amount ranges from 1 nM to 20 nM, such as 4-6 nM to 10 nM and including 4 nM to 8 nM, where in some instances it may be 1 nM, 2 nM, 3 nM, 4 nM, 5 nM, 6 nM, 7 nM, 8 nM, 9 nM or 10 nM. While any suitable binding conditions may be employed, in some instances the conditions may include incubating a binding reaction mixture that includes the normalization binding moiety and the nucleic acid sample as a buffered reaction mixture (e.g., a reaction mixture buffered with Tris-acetate, or the like) at a pH of from 7 to 8, such as pH 7.5, under suitable temperatures, such as from 32° C. to 42° C., such as 37° C. The binding reaction may be allowed to proceed for a sufficient amount of time, such as from 5 minutes to 3 hours. The binding reaction results in the production of binding complexes that include a normalization binding moiety and a nucleic acid having a common target to which the normalization binding moiety specifically binds. As reviewed above, as the normalization binding moiety is employed in a limiting amount, not all of the nucleic acids of the sample may be present in a binding complex. Instead, the resultant composition may include binding complexes as well as excess, unbound nucleic acids and any other entities that may be present that do not bind to the normalization binding moiety, e.g., primer dimers, etc.

Resultant complexes comprising normalization binding moieties bound to nucleic acids can then be separated from unbound nucleic acids of the binding reaction mixture. Any suitable separation strategy may be employed. Such strategies may include separating the complexes from other constituents of the composition (e.g., through purification, such as using spin column purification). In certain aspects, the normalization binding protein includes a purification domain, e.g., tag (e.g., an epitope tag), and the binding complexes may be separated from other constituents by affinity purification. For example, the binding complexes may be immobilized on the surface of a solid phase (e.g., a column, a plate, beads (e.g., agarose or magnetic beads), and/or the like) that includes a binding partner of the tag (e.g., an antibody or other suitable binding partner that binds the tag), and then washed to remove any residual constituents of the composition. Spin columns can be configured to separate the normalization binding protein from the library sample (as described in US publication Number 2015023890, herein incorporated by reference in its entirety).

A spin column can include an elongated hollow structure having a sample inlet at a first end and a sample outlet at a second end; and a poly(acid) membrane matrix positioned in the elongated hollow structure such that fluid must flow through the poly(acid) membrane to traverse the structure from the first end to the second end. The poly(acid) membrane matrix may vary. In some instances, the poly(acid) membrane matrix Includes a poly(acid) component adsorbed to a surface of a porous membrane support. The poly(acid) component may have a variety of configurations on the surface of the porous membrane component. For example, the poly(acid) component may be arranged as a film, e.g., coating or layer (including layer by layer) configuration on the surface of the porous membrane. Alternatively, the poly(acid) component may be configured as a plurality of polymeric brushes on a surface of the porous membrane. The surface of the porous membrane may be any surface, including an upper surface, the surface of the pores of the membrane, etc., where in some instances all surfaces of the membrane may be stably associated with, e.g., adsorbed to, the poly(acid) component. In certain embodiments, poly(acid) films configured in a layer-by-layer configuration may be configured in a heteropolymer coating or a heteropolymer layer-by-layer configuration. Heteropolymer layer-by-layer configurations are those poly(acid) films that may be composed of two or more different heteropolymers. Heteropolymer layer-by-layer configurations also include those poly(acid) films that may be composed of at least two different species of homopolymers, i.e., a hetero-homopolymer. Where desired, the poly(acid) matrix may further include an affinity element. The affinity element is an element or component that displays binding affinity for a category of molecules or a specific molecule. Affinity elements may be, in some cases defined as non-specific affinity elements, e.g., those affinity elements that bind a category of molecules, or, in some instances, may be defined as specific affinity elements, e.g., those affinity elements that bind a specific molecule. In some instances, the affinity element on the polyacid film or affinity purification system can bind to the purification tag on the normalization binding protein. Exemplary affinity elements can include, a metal ion chelating ligand complexed with a metal ion which, e.g., which binds to any suitable tagged protein in a given sample. The metal ion chelating ligand complexed with a metal ion may vary with respect to the ligand and the metal ion. Examples of ligands of interest include, but are not limited to: iminodiacetic acid (IDA), nitriloacetic acid (NTA), caboxymethylated aspartic acid (CM-Asp), tris(2-aminoethyl)amine (TREN), and tris-carboxymethyl ethylene diamine (TED). These ligands offer a maximum of tri-(IDA), tetra-(NTA, CM-Asp), and penta-dentate (TED) complexes with the respective metal ion. A variety of different types of metal ions may be complexed to the ligands of the subject compounds. Metal ions of interest can be divided into different categories (e.g., hard, intermediate and soft) based on their preferential reactivity towards nucleophiles. Hard metal ions of interest include, but are not limited to: Fe³⁺, Ca²⁺ and Al³⁺ and like. Soft metal ions of interest include, but are not limited to: Cu+, Hg²+, Ag+, and the like.

Intermediate metal ions of interest include, but are not limited to: Cu²⁺, Ni²⁺, Zn²⁺, Co²⁺ and the like. In certain embodiments, the metal ion that is chelated by the ligand is Co²⁺. In certain embodiments, the metal ion of interest that is chelated by the ligand is Fe³⁺. Additional metal ions of interest include, but are not limited to lanthanides, such as Eu³⁺, La³⁺, Tb³⁺, Yb³⁺, and the like. In certain embodiments, the affinity element includes aspartate groups and is referred to as an aspartate-based metal ion affinity element, where such compositions include a structure that is synthesized from an aspartic acid, e.g., L-aspartic acid. Aspartate-based metal ion affinity elements include aspartate-based ligand/metal ion complexes, e.g., tetradentate aspartate-based ligand/metal ion complexes, where the metal ion complexes have affinity for proteins, e.g., proteins tagged with a metal ion affinity peptide. In some instances, aspartate-based compositions of the present disclosure include structures having four ligands capable of interacting with, i.e., chelating, a metal ion, such that the metal ion is stably but reversibly associated with the ligand, depending upon the environmental conditions of the ligand. In some instances, the tag-binding affinity element may be a polypeptide, e.g., an antibody, that directly binds the polypeptide epitope tag, e.g., an anti-FLAG antibody. Antibodies that bind polypeptide epitope tags include but are not limited to: anti-FLAG antibodies, anti-His epitope tag antibodies, anti-HA tag antibodies, anti-Myc epitope tag antibodies, anti-GST tag antibodies, anti-GFP tag antibodies, anti-V5 epitope tag antibodies, anti-6x His tag antibodies, anti-6xHN tag antibodies, and the like. Such antibodies are available from commercial suppliers, e.g., from Takara Bio USA (Mountain View, Calif.), Thermo Scientific (Rockford, Ill.), and the like.

In some instances, following separation of the binding complexes, the nucleic acids of the binding complexes may then be disassociated from the normalization binding moieties of the binding complexes (e.g., to produce normalized libraries for, e.g., sequencing).The nucleic acids of the binding complexes may be recovered from the normalization binding moieties using a suitable elution buffer (e.g., a buffer that, for example, includes a protein denaturation agent, such as sodium dodecyl sulfate (SDS)), using a buffer that includes a reagent that digests the nuclease component (e.g., proteinase K), using heat denaturation, using DTT or betamercaptoethanol to break S-S bonds, and/or the like, to disrupt the interactions between the nucleic acids and the normalization binding moieties. Approaches for affinity purification and recovering nucleic acids from protein complexes are described, e.g., in Methods for Affinity-Based Separations of Enzymes and Proteins (Munishwar Nath Gupta, ed., Birkhäuser Verlag, Basel-Boston-Berlin, 2002); Chromatin Immunoprecipitation Assays: Methods and Protocols (Collas, ed., 2009); and The Protein Protocols Handbook (Walker, ed., 2002). If desired, the separated target nucleic acids may be further purified by alcohol precipitation, column purification, gel purification, or any other convenient nucleic acid purification strategy, e.g., as described below.

In some instances, the nucleic acids of the binding complexes may then be disassociated from the normalization binding moieties of the binding complexes may be performed during the above described separation step, e.g., so as to reduce the number of steps in the overall workflow.

Depending on the particular protocol, resultant normalized amounts of nucleic acids obtained from each nucleic acid sample may then be combined or pooled, as desired. For example, molecules across multiple libraries can be pooled for sequencing. In yet other embodiments, the two or more nucleic acid samples are pooled prior to normalization, e.g., as described above. For example, sample barcodes may be employed as common targets for normalization in accordance with embodiment of the invention. In this case, two or more libraries, e.g., as described above, may be pooled together before normalization and then normalized in parallel by using a pooled normalization binding moiety composition that is made up of limiting amounts of different normalization binding moieties that are specific for each target nucleic population of interest. For example, where the normalization binding moiety is a Cas9/sgRNA, the pooled normalization binding moiety composition may be made up of multiple different Cas9/guides each specific to the specific barcode of one of the libraries and each at the same limiting concentration, so that all the libraries are normalized simultaneously together as a single pool. Such embodiments find using in many situations, including the normalization of single cell libraries, which are typically pooled such that there is no way to normalize by current methods. For example, a set of single cell libraries may be fabricated using the icell8 system (Takara Bio USA, Mountain View, Calif.) where the cells are then normalized to each other from the pool taken off the icell8 chip. Where desired, one can then further normalize this set of libraries to another set by using a normalization strategy targeting adapter sequence, e.g., cas9/guide normalization strategy targeting the adapter sequences—e.g., p5/p7, such as described above, for two or more sets of single cell libraries.

The methods may be performed in any suitable environment. In some instances, the methods are performed in a single container. Single containers of interest include tubes, plates, wells of multi-well arrays, and droplets, or any combination thereof.

In this way, the disclosure provides methods for normalizing across different libraries to allow evenly distributed read depth across samples.

### Nucleic Acid Samples

The method may be employed to normalize a variety of different types of nucleic acid samples. As reviewed above, nucleic acid samples that may be normalized in accordance with embodiments of the invention may vary, where in some instances the nucleic acid samples are compositions made up of a plurality of distinct nucleic acids that differ from each other in terms of overall sequence. While the number of distinct nucleic acids (e.g., deriving from distinct genes) in a given nucleic acid sample may vary, in some instances the number of distinct nucleic acids present in a given nucleic acid sample is 10 or more, such as 25 or more, 50 or more, 100 or more, 500 or more, 1000 or more, 10,000 or more, 20,000 or more, where in some instances the number of distinct nucleic acids ranges from 1,000 to 25,000, such as 2,000 to 20,000. The nucleic acid constituents of a given nucleic acid sample may be single stranded nucleic acids or double stranded nucleic acids, where in some instances the nucleic acids are double stranded deoxyribonucleic acids (dsDNAs). A variety of different types of nucleic acid samples may be normalized according to embodiments of the inventions, where examples of nucleic acid samples that may be normalized include, but are not limited to: next generation sequencing (NGS) libraries, microarray libraries, etc. Further details regarding types of libraries that may be normalized in accordance with methods of the invention, including the preparation thereof, are provided below. Nucleic acid samples that are normalized in accordance with embodiments of the invention may be obtained from a variety of sources, such as but not limited to: a single cell, a plurality of cells (e.g., cultured cells), a tissue, an organ, or an organism (e.g., mouse, rat, or the like). In some instances, a nucleic acid samples that are normalized by methods of the invention may be derived from a cellular sample, such as a sample containing 10 cells or less, including a single cell sample. As used herein, a “single cell” refers to one cell. Single cells useful as the source of template RNAs and/or in generating single cell libraries, such as expression libraries and/or immune cell receptor repertoire libraries can be obtained from a tissue of interest, or from a biopsy, blood sample, or cell culture. Additionally, cells from specific organs, tissues, tumors, neoplasms, or the like can be obtained and used in the methods described herein. In certain aspects, the initial nucleic acid sample is obtained from a cell(s), tissue, organ, and/or the like, including but no limited to: embryos, blastocysts, spent media, culture media, blood, fresh fixed frozen tissues, etc. In some aspects, the initial nucleic acid sample is obtained from a mammal (e.g., a human, a rodent (e.g., a mouse), or any other mammal of interest). In other aspects, the nucleic acid sample is isolated from a source other than a mammal, such as amphibians (e.g., frogs (e.g., Xenopus)), fish (zebrafish (Danio rerio), or any other non-mammalian nucleic acid sample source, e.g., plants, bacteria, viruses, fungi, etc.

In some instances, the nucleic acid samples are next generation sequencing libraries. Next generation sequencing (NGS) libraries are collections of nucleic acids, e.g., as described above, where the nucleic acid members include a templated sequence and one or more non-templated sequences. A templated sequence is a sequence that corresponds to a template nucleic acid and templated by a template, e.g., a RNA (such as mRNA) or DNA (such a genomic DNA) template. The terms “non-templated sequence” and “non-template sequence” generally refer to those sequences that do not correspond to a template (e.g., are not present in templates, do not have a complementary sequence in a template or are unlikely to be present in or have a complementary sequence in a template). Non-templated sequences are those that are not templated by a template, e.g., a RNA or DNA template, and thus they may be, e.g., added during an elongation reaction in the absence of corresponding template, e.g., nucleotides added by a polymerase having non-template directed terminal transferase activity. The addition of non-templated sequence to a nucleic acid need not be necessarily limited to elongation reaction. For example, in some instances, a non-templated sequence may be added through ligation of the non-templated sequence to the nucleic acid, through a transposase mediated reaction, e.g., through a tagmentation reaction which adds the non-templated sequence to a subject nucleic acid, etc. Nucleic acid libraries that may be normalized according to embodiments of the methods may vary, where examples include, but are not limited to, those made by tagmentation, those made by ligation, those made by PCR, e.g. ThruPLEX libraries or AmpliSeq libraries, libraries made by other ligation methods such as ULTRA II from NEB, libraries made using Tru-Seq adapters/Y adapters, libraries made using template switch oligonucleotide (TSO) mediated protocols, etc.

In some instance, the non-templated portion(s) of the nucleic acid constituents of the library may include partial or complete sequencing platform adapter sequences, such that the nucleic acid members of a given NGS library to be normalized by methods of the invention may include at one or both of their termini partial or complete sequencing platform adapter sequences useful for sequencing using a sequencing platform of interest. Sequencing platforms of interest include, but are not limited to, the HiSeg™, MiSeg™ and Genome Analyzer™ sequencing systems from Illumina®; the Ion PGM™ and Ion Proton™ sequencing systems from Ion Torrent™; the PACBIO RS II Sequel system from Pacific Biosciences, the SOLiD sequencing systems from Life Technologies™, the 454 GS FLX+ and GS Junior sequencing systems from Roche, the MinION™ system from Oxford Nanopore, or any other sequencing platform of interest.

In some instances, a non-templated sequence, e.g., present on an oligonucleotide and/or a nucleic acid primer, includes a sequencing platform adapter construct. By “sequencing platform adapter construct” is meant a nucleic acid construct that includes at least a portion of a nucleic acid domain (e.g., a sequencing platform adapter nucleic acid sequence) or complement thereof utilized by a sequencing platform of interest, such as a sequencing platform provided by Illumina® (e.g., the HiSeq™, MiSeq™ and/or Genome Analyzer™ sequencing systems); Ion Torrent™ (e.g., the Ion PGM™ and/or Ion Proton™ sequencing systems); Pacific Biosciences (e.g., the PACBIO RS II sequencing system); Life Technologies™ (e.g., a SOLiD sequencing system); Roche (e.g., the 454 GS FLX+ and/or GS Junior sequencing systems); or any other sequencing platform of interest.

Adapter constructs attached to the ends of a nucleic acid of interest or a derivative thereof may include any sequence elements useful in a downstream sequencing application. For example, the adapter constructs attached to the ends of nucleic acid of interest or a derivative thereof may include a nucleic acid domain or complement thereof selected from the group consisting of: a domain that specifically binds to a surface-attached sequencing platform oligonucleotide, a sequencing primer binding domain, a barcode domain, a barcode sequencing primer binding domain, a molecular identification domain, and combinations thereof. Where desired, the one or more of these domains may include a common target sequence to which the normalization binding moiety specifically binds. In yet other instances, an additional domain that includes the common target sequence may be present. A nucleic acid domain, such as described above, refers to a stretch or length of a nucleic acid made up of a plurality of nucleotides, where the stretch or length provides a defined function to the nucleic acid. In some instances, the terms “domain” and “region” may be used interchangeably, including e.g., where immune receptor chain domains/regions are described, such as e.g., immune receptor constant domains/regions. While the length of a given domain may vary, in some instances the length ranges from 2 to 100 nt, such as 5 to 50 nt, e.g., 5 to 30 nt.

In certain aspects, a non-templated sequence includes a sequencing platform adapter construct that includes a nucleic acid domain that is a domain (e.g., a “capture site” or “capture sequence”) that specifically binds to a surface-attached sequencing platform oligonucleotide (e.g., the P5 or P7 oligonucleotides attached to the surface of a flow cell in an Illumina® sequencing system); a sequencing primer binding domain (e.g., a domain to which the Read 1 or Read 2 primers of the Illumina® platform may bind). The sequencing platform adapter constructs may include nucleic acid domains (e.g., “sequencing adapters”) of any length and sequence suitable for the sequencing platform of interest. In certain aspects, the nucleic acid domains are from 4 to 200 nts in length. For example, the nucleic acid domains may be from 4 to 100 nts in length, such as from 6 to 75, from 8 to 50, or from 10 to 40 nts in length. According to certain embodiments, the sequencing platform adapter construct includes a nucleic acid domain that is from 2 to 8 nts in length, such as from 9 to 15, from 16-22, from 23-29, or from 30-36 nts in length.

The nucleic acid domains may have a length and sequence that enables a polynucleotide (e.g., an oligonucleotide) employed by the sequencing platform of interest to specifically bind to the nucleic acid domain, e.g., for solid phase amplification and/or sequencing by synthesis of the cDNA insert flanked by the nucleic acid domains. Example nucleic acid domains include the P5 (5′-AATGATACGGCGACCACCGA-3′)(SEQ ID NO:01), P7 (5′-CAAGCAGAAGACGGCATACGAGAT-3′)(SEQ ID NO:02), Read 1 primer (5′-ACACTCTTTCCCTACACGACGCTCTTCCGATCT-3′)(SEQ ID NO:03) and Read 2 primer (5′-GTGACTGGAGTTCAGACGTGTGCTCTTCCGATCT-3′)(S EQ ID NO:04) domains employed on the Illumina®-based sequencing platforms. Other example nucleic acid domains include the A adapter (5′-CCATCTCATCCCTGCGTGTCTCCGACTCAG-3′)(SEQ ID NO:05) and P1 adapter (5′-CCTCTCTATGGGCAGTCGGTGAT-3′)(SEQ ID NO:06) domains employed on the Ion Torrent™-based sequencing platforms.

The nucleotide sequences of non-templated sequence domains useful for sequencing on a sequencing platform of interest may vary and/or change over time. Adapter sequences are typically provided by the manufacturer of the sequencing platform (e.g., in technical documents provided with the sequencing system and/or available on the manufacturer's website). Based on such information, the sequence of the sequencing platform adapter construct of the non-templated sequence (e.g., a template switch oligonucleotide and/or a single product nucleic acid primer, and/or the like) may be designed to include all or a portion of one or more nucleic acid domains in a configuration that enables sequencing the nucleic acid insert (corresponding to the template nucleic acid) on the platform of interest. Sequencing platform adaptor constructs that may be included in a non-templated sequence as well as other nucleic acid reagents described herein, are further described in U.S. patent application Ser. No. 14/478,978 published as US 2015-0111789 A1, the disclosure of which is herein incorporated by reference.

Non-templated sequence may be added to a nucleic acid of interest, e.g., to an oligonucleotide, a nucleic acid primer, a generated dsDNA, etc., by a variety of means. For example, as noted above, non-templated sequence may be added through the action of a polymerase with terminal transferase activity. Non-templated sequence, e.g., present on a primer or oligonucleotide, may be incorporated into a product nucleic acid during an amplification reaction. In some instances, non-templated nucleic acid sequence may be directly attached to a nucleic acid, e.g., to a primer or oligonucleotide prior to amplification, to a product of nucleic acid amplification, etc. Methods of directly attaching a non-templated sequence to a nucleic acid will vary and may include but are not limited to e.g., template switching, ligation, chemical synthesis/linking, enzymatic nucleotide addition (e.g., by a polymerase with terminal transferase activity), and the like.

Additional examples of NGS libraries and methods of preparing the same which may be normalized using methods of the present invention include, but are not limited to, those described in United States Patent Application Publication Nos. 20150111789, 20170198285, 20170198284, 20150203906, 20170327882, 20190010489, 20160304935, 20190112648, 20030064376, 20030143599, 20040209298, 20130085083, 20050202490, 20070031857, 20150284712, 20160257985 and 20160289723, as well as published PCT application Publication Nos. WO 2018/089550, WO 2018/152129 and WO 2019/040788, the disclosures of which are herein incorporated by reference.

### Sequencing

In certain embodiments, the provided methods further include subjecting the normalized nucleic acid samples to a sequence protocol, such as an NGS protocol. The protocol may be carried out on any suitable NGS sequencing platform. NGS sequencing platforms of interest include, but are not limited to, a sequencing platform provided by Illumina® (e.g., the HiSeg™, MiSeg™ and/or NextSeg™ sequencing systems); Ion Torrent™ (e.g., the Ion PGM™ and/or Ion Proton™ sequencing systems); Pacific Biosciences (e.g., the PACBIO RS II Sequel sequencing system); Life Technologies™ (e.g., a SOLiD sequencing system); Oxford Nanopore (e.g., MinION), Roche (e.g., the 454 GS FLX+ and/or GS Junior sequencing systems); or any other sequencing platform of interest. The NGS protocol will vary depending on the particular NGS sequencing system employed. Detailed protocols for sequencing an NGS library, e.g., which may include further amplification (e.g., solid-phase amplification), sequencing the amplicons, and analyzing the sequencing data are available from the manufacturer of the NGS sequencing system employed.

### Cleanup

At one or more steps of the methods, e.g., during nucleic acid sample (such as library) preparation, following normalization and prior to pooling, etc., a cleanup step may be performed wherein sample constituents, e.g., primers not extended along a nucleic acid template, primer dimers, etc., are preferentially removed or depleted from a sample. Optionally, this step may be performed by a gel-based size selection step. Optionally, this size selection step may be performed with a solid-phase reversible immobilization process, such as a size selection step involving magnetic or superparamagnetic beads. Optionally, this size selection step may be performed with a column-based nucleic acid purification or size-selection step. Optionally, this size selection step may remove nucleic acid molecules less than 50 nucleotides in length, less than 100 nucleotides in length, less than 150 nucleotides in length, less than 200 nucleotides in length, less than 300 nucleotides in length, less than 400 nucleotides in length, less than 500 nucleotides in length, or less than 1000 nucleotides in length.

In some instances, non-sequence specific proteinaceous binding agents may be employed in a reversible immobilization process. Non-specific proteinaceous binding agents include non-specific DNA binding proteins, such as but not limited to: structural proteins, e.g. histones, high-mobility group (HMG) proteins, etc. Where desired, these non-sequence specific proteinaceous binding agents may be present on the surface of a solid support, e.g., magnetic or superparamagnetic beads, etc. While this embodiment of size selection cleanup is disclosed in the context of library normalization methods, it is not limited to use in such, but may find use in any nucleic acid sample preparation protocol where a cleanup step is desired. As such, this cleanup protocol may be employed in workflows that do not include a nucleic acid sample normalization step as described herein.

### Compositions and Kits

Aspects of the present disclosure also include compositions and kits. The compositions and kits may include, e.g., one or more of any of the reaction mixture components described above with respect to the subject methods. For example, the compositions and kits may include a normalizing binding moiety, e.g., a normalization binding protein, e.g., as described above. In addition, the compositions and kits may include one or more additional components that find use practicing embodiments of the invention, where such additional components include, but are not limited to: purification/separation components, cleanup components, nucleic acid sample, e.g., NGS library, preparation components, such as primers, including tagged primers, etc. Components of the kits may be present in separate containers, or multiple components may be present in a single container.

In addition to the above-mentioned components, a subject kit may further include instructions for using the components of the kit, e.g., to practice the subject methods as described above. In addition, the kit may further include programming for analysis of results including, e.g., counting unique molecular species, etc. The instructions and/or analysis programming may be recorded on a suitable recording medium. The instructions and/or programming may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD) etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following example is offered by way of illustration and not by way of limitation.

### EXAMPLES

### Example 1: Method of Normalizing Libraries Using Limiting Amounts of Cas9

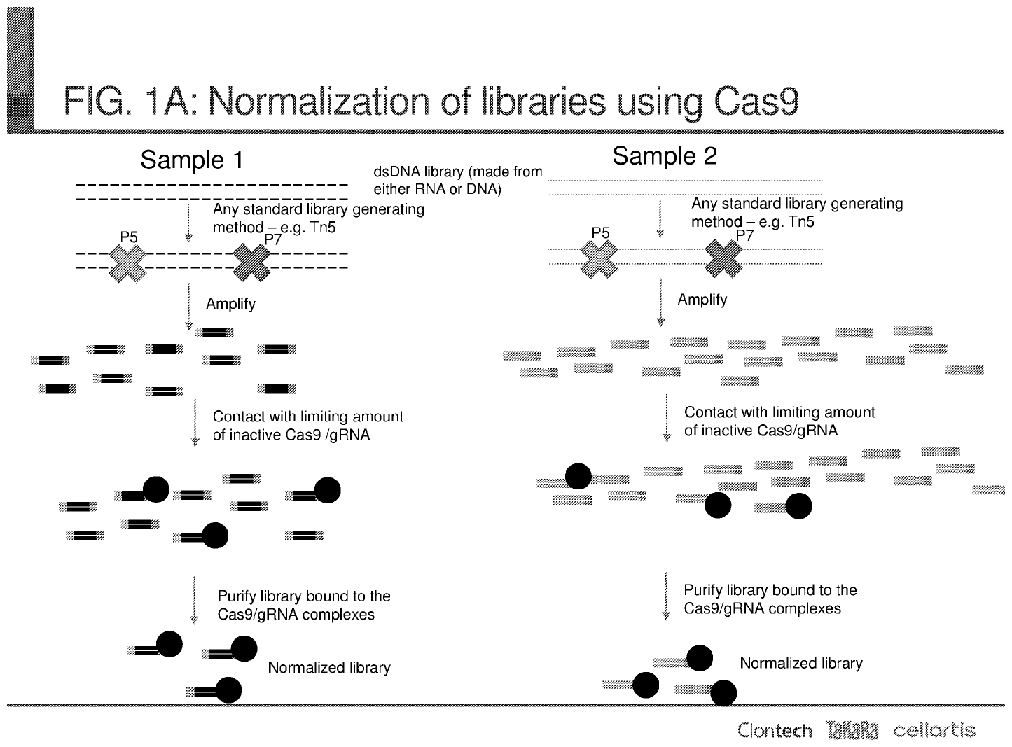

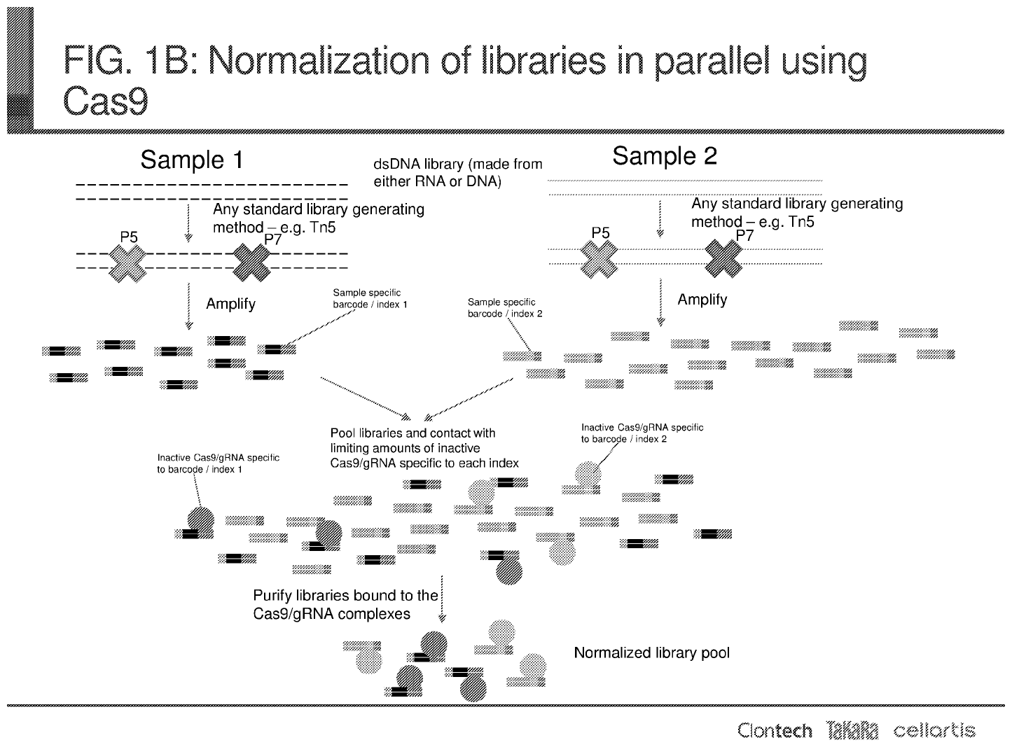

This example describes a method for normalizing libraries using limiting amounts of an inactive Cas9 as a normalization binding protein as shown in FIG. 1A. Sequencing libraries from two or more samples are generated. The libraries originate from RNA or DNA. To generate the libraries of target fragments from the source RNA or DNA, any standard method of library construction for next generation sequencing known in the art can be employed so as to add appropriate sequencing adapters, including Illumina p5 and p7 sequences to the target fragments, for example: “A-tailing” and adapter ligation such as used in Illumina's tru-seq DNA library prep kits, tagmentation as used in Illumina's Nextera kits, direct amplification as used in amplicon panels, such as Illumina's AmpliSeq for Illumina Cancer Hotspot Panel v2 or template switching such as TBUSA's SMARTer kits for RNA sequencing. The library fragments thereby have common end sequences (e.g., P5 and P7). The libraries are then contacted with a limiting amount of inactive Cas9 protein. In some cases, genetically inactivated Cas9 protein, such as dCas9, is used. In some other cases, the Cas9 protein is inactivated by modulating the buffer conditions. For example, the Cas9 protein can be inactivated by adding chelating agents, such as EDTA, to the reaction mixture. The inactive Cas9 protein comprises an affinity tag (e.g., his-tag). The inactive Cas9 protein comprises a guide RNA (gRNA) to form a Cas9/gRNA complex. The Cas9/gRNA complex is designed to hybridize to one of the common ends (e.g., P5 or P7) and/or to any common sequence between the samples, such as transposable element sequences in case of the tagmented libraries. The specific hybridization of Cas9/gRNA complex to the library molecules eliminates primer-dimers from the reaction mixture. The library bound to the Cas9/gRNA complex is passed through a spin column comprising a membrane comprising a his-tag binding element (e.g., Ni²⁺-NTA). The library bound to the Cas9/gRNA complex, in turn, binds to the column, thereby bringing with it the bound library molecules. The library molecules are eluted with or without the Cas9/gRNA complex from the column. Alternatively, the Cas9/gRNA complex is mixed with magnetic beads, so that the complexes bind to the beads. Unbound materials are then removed, and the beads washed, before eluting the library molecules from the beads with or without the Cas9/gRNA complex. In some instances, the library molecules are disassociated from the Cas9/gRNA complex prior to sequencing the library. In some cases, additional steps, for example to remove inhibitors (e.g., EDTA), can be included prior to sequencing the library. The library molecules are pooled between samples and sequenced on a sequencer in a sequencing reaction.

### Example 2: Method of Normalizing Libraries Using Limiting Amounts of Tagged Primers

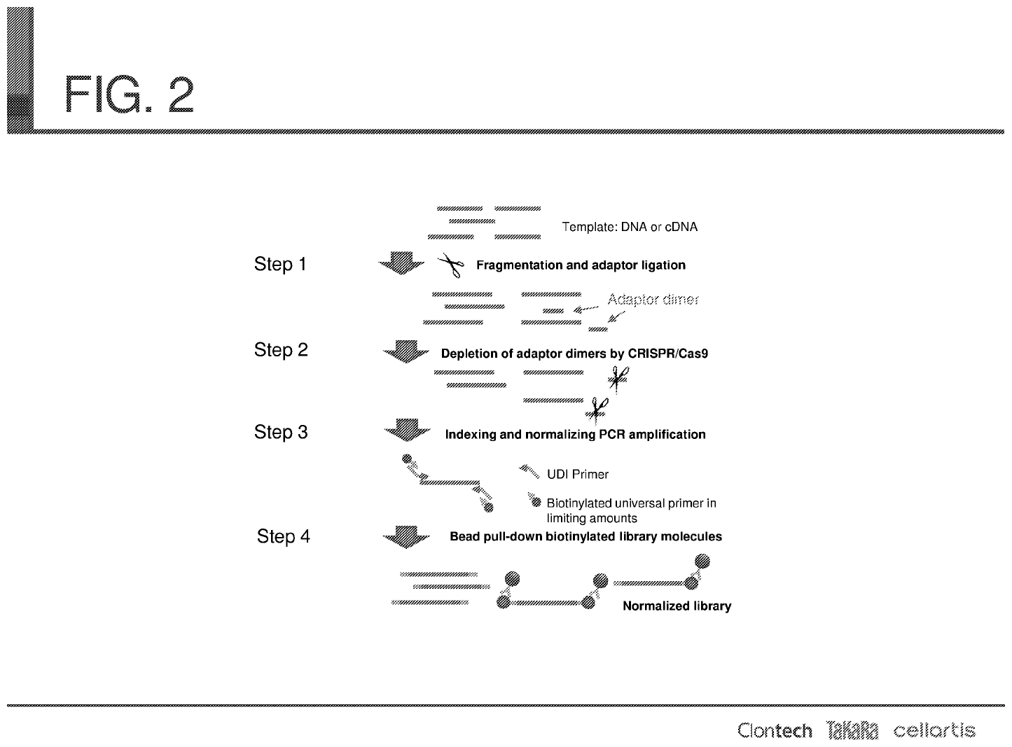

This example describes a method for normalizing libraries using limiting amounts of tagged primers, such as biotinylated primers, as shown in FIG. 2. Sequencing libraries from two or more samples are generated. The libraries originate from RNA or DNA. To generate the libraries of target fragments from the source RNA or DNA, any standard method of library construction for next generation sequencing known in the art can be employed so as to add appropriate sequencing adapters, including Illumina p5 and p7 sequences to the target fragments, for example: “A-tailing” and adapter ligation such as used in Illumina's tru-seq DNA library prep kits, tagmentation as used in Illumina's Nextera kits, direct amplification as used in amplicon panels, such as Illumina's AmpliSeq for Illumina Cancer Hotspot Panel v2 or template switching such as TBUSA's SMARTer kits for RNA sequencing. Primer-dimers or adapter dimers generated during the library construction are eliminated using any one or more of the methods described in Example 5. The libraries are then amplified with a limiting amount of tagged common primers, such as biotinylated primers, thereby generating normalized biotinylated libraries. The resultant libraries are then pulled down using streptavidin coated beads or a streptavidin column (e.g., Capturem™ Streptavidin Miniprep Columns) to obtain normalized libraries. The libraries can be removed from the beads or column by any standard process known in the art for removing materials from columns or beads including for example, heat denaturation of the bound protein, enzymatic or chemically-induced cleavage, adjustment to salt or pH. In some instances, the libraries are separated from the beads or column using the addition of free biotin. In the presence of free biotin, streptavidin dissociates from biotinylated dsDNA, as described in the below thesis: https://scholarcolorado.edu/cgi/viewcontent.cgi?article=2604&context=honr_theses. The library molecules are pooled between samples and sequenced.

### Example 3: Method of Normalizing Libraries Using Limiting Amounts of Inactive Transposase

This example describes a method of normalizing libraries using limiting amounts of an inactive Transposase, Tn5, for example. As described in Example 1, sequencing libraries from two or more samples are generated. The libraries originate from RNA or DNA. The libraries are tagmented with Tn5 transposon complexes comprising either P5 or P7 Illumina flow cell adaptors. In some instances, P5 or P7 Illumina flow cell adaptors can be added in a separate PCR reaction rather than during tagmentation. The tagmented libraries, comprising transposable element (also referred to as TE) sequences, are contacted with a limiting amount of inactivated transposase enzyme which cannot carry out the transposition reaction and instead just binds to the library molecules, e.g., at the TE sequences in the tagmented libraries. If the inactive transposase specifically binds to the TE sequences in the library molecules, then the presence of primer-dimers in the library can be avoided. The inactive transposase binds to the library molecules forming a complex. In some cases, genetically inactivated transposase is used while in other cases, chemically inactivated transposase is used, for example by exposing the enzyme to EDTA. The inactive transposase enzyme comprises an affinity tag (e.g., his-tag). The sample is passed through a spin column comprising a membrane comprising a his-tag binding element (e.g., Ni²⁺-NTA), such as Capturem™ His-Tagged Purification Miniprep Kit. The transposase in the complex binds to the column, thereby bringing with it the bound library molecules. The library molecules are eluted with or without the inactive transposase from the column. Alternatively, the sample is mixed with magnetic Ni²⁺-NTA beads, so that the transposase/library complexes bind to the beads. Unbound materials are then removed, and the beads washed, before eluting the library molecules from the beads with or without the transposase. The library molecules are pooled between samples and sequenced.

### Example 4: Method of Normalizing Libraries Using Membranes with Limiting Capacity of Binding to Library Molecules

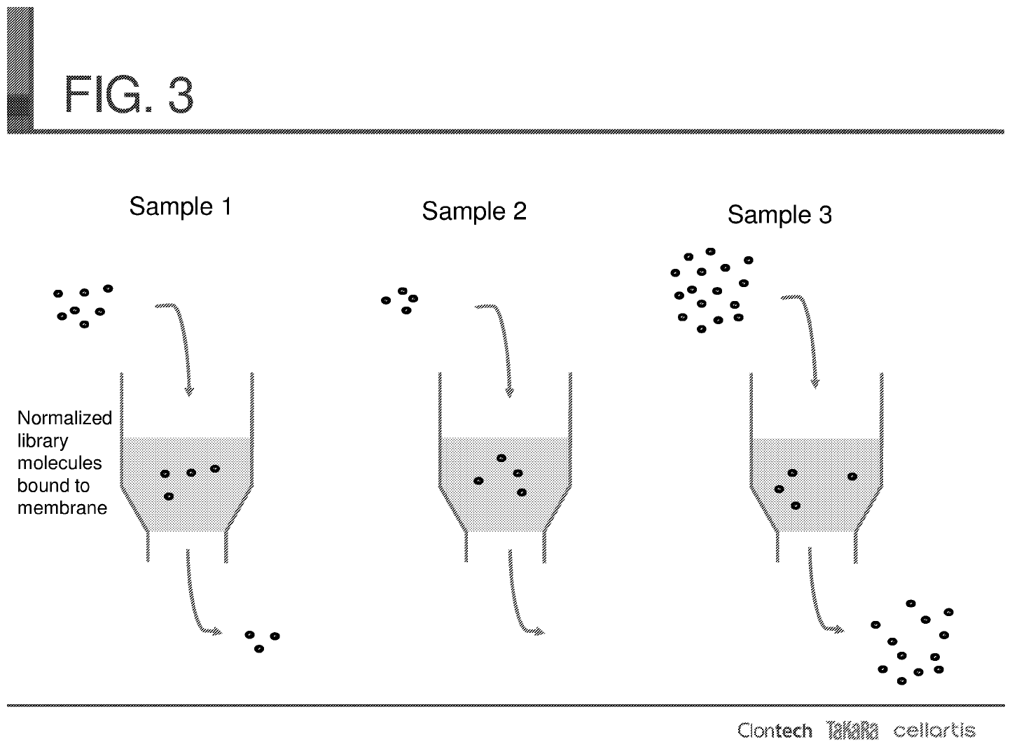

This example describes a method for normalizing libraries using membranes with limited capacity of binding to the library molecules. The libraries originate from RNA or DNA. The libraries are fragments and ligated to either P5 or P7 Illumina flow cell adaptors. Primer-dimers are eliminated using any one or more of the methods described in Example 5. In some cases, membranes with carboxyl groups, such as poly(acid) membranes having a limited capacity of binding to the library molecules, are used. For example, as illustrated in FIG. 3, Takara's Capturem® products comprising poly(acid) membranes are used for library normalization. Each of the library sample is passed through a Capturem® column to generate normalized libraries. In some instances, the amount of sample passed through the membrane is greater than the binding capacity of the membrane. In other words, the membrane is loaded with a saturating amount of the library. In yet other instances, the libraries are passed through the membranes under specific conditions, such as in the presence of crowding agents (e.g., polyethylene glycol) in order to modulate binding of the library to the membrane, such that a fixed amount is able to bind. The bound library molecules are thus normalized in amount and can then be eluted. The eluted, normalized library molecules are pooled between samples and sequenced.

### Example 5: Methods for Eliminating Primer-dimers from the Sequencing Libararies

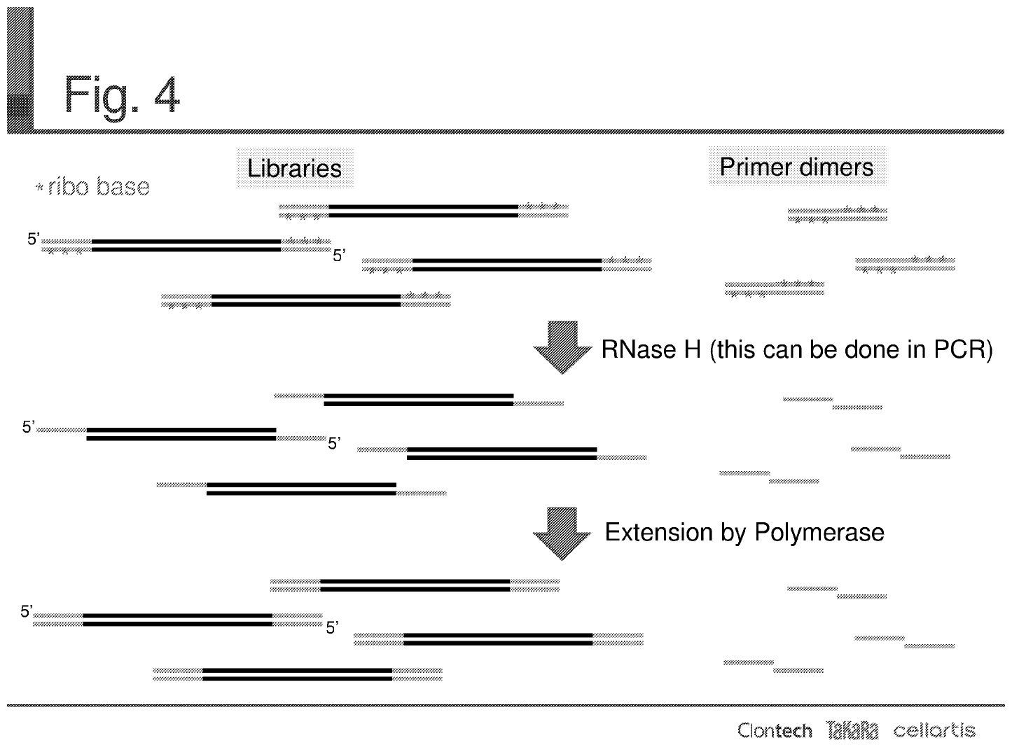

In some cases, primer-dimers or adapter dimers are eliminated using CRISPR/Cas9 by designing one or more guide RNA to the junction of adapter ligation e.g., as described in: https://www.ncbi.nlm.nih.gov/pubmed/31165880. This removal step may be done either before, after or during the normalization step, as may be convenient to the user. In some other instances, a suppression PCR is used to eliminate primer-dimers (Suppression PCR is described here: Gurskaya N G, Diachenko L, Chenchik A, Siebert P D, Khaspekov G L, Lukyanov K, Vagner L L, Ermolaeva O D, Lukyanov S, Sverdlov E D. (1996) The Equalizing cDNA Subtraction Based on Selective Suppression of Polymerase Chain Reaction: Cloning of the Jurkat Cells' Transcripts Induced by Phytohemaglutinin and Phorbol 12-myristate 13-acetate. Anal. Biochem. 240(1): 90-97./PMID:8811883). For example, a PCR assay are designed such that the primers will only amplify the library molecules and not the primer-dimers. In some other instances, size selection methods can be employed to eliminated primer-dimers. For example, magnetic beads selectively binding to the library molecules are used to retain the library molecules on the beads while eluting the primer-dimers. In some other instances, at least one cleavable base is included in the primers during the steps for generating the library molecules. As shown in FIG. 4, the library is then treated with a cleavage agent, including but not limited to for example RNase H or USER® enzyme mix from New England Biolabs, so as to cleave the at least one cleavable base in the primers. In some cases, the cleavage agent is included in the subsequent step to reduce the number of steps in the workflow. This step results in a library with single-stranded primer sequences ligated to each end of the library molecules and the primer-dimers, mostly single-stranded, with some or no hybridized regions. The library is then treated with a polymerase to generate library molecules with double-stranded primer sequences on both ends of the molecule while eliminating the primer-dimers from the mixture.

### Example 6: Method of Normalizing Libraries in Parallel Using Limiting Amounts of Cas9

This example is an extension of the method described in Example 1 and is also outlined in FIG. 1 B. In Example 1, samples are normalized independently and pooled together after normalization. In this example, the libraries are normalized in parallel. This is done by designing guide RNAs that are specific for each library rather than for every library—for example, by designing the guide RNAs against the barcode or index sequences in the library adapters. Since each index/barcode is specific to a given library/sample, the guide RNA will thus target the inactive Cas9 to only those library fragments carrying the specific barcode of the particular sample. Limiting amounts of guide RNA are generated representing each of the sample barcodes in the pool. These are combined with inactive cas9 (dCas9) and mixed with the pooled libraries. The fragments from each library bind to each respective limiting about of dCas9/guideRNA complex. The pool libraries bound to the Cas9/g RNA complexes is passed through a spin column comprising a membrane comprising a his-tag binding element (e.g., Ni²⁺-NTA). The library bound to the Cas9/g RNA complex, in turn, binds to the column, thereby bringing with it the bound library molecules. The library molecules are eluted with or without the Cas9/gRNA complex from the column. Alternatively, the Cas9/gRNA complex is mixed with magnetic beads, so that the complexes bind to the beads. Unbound materials are then removed, and the beads washed, before eluting the library molecules from the beads with or without the Cas9/gRNA complex. In some instances, the library molecules are disassociated from the Cas9/gRNA complex prior to sequencing the library. In some cases, additional steps, for example to remove inhibitors (e.g., EDTA), can be included prior to sequencing the library. The eluted pool contains normalized amounts of each of the libraries.

### Example 7: Method of Normalizing Molecules Within a Single Library

In Example 6, FIG. 1B is shown a method for normalizing several libraries in parallel by using Cas9/guide RNA complexes specific to each library's barcode sequence. In some cases, it may be advantageous to normalize the levels of the various individual fragments in a single library, for example, if one is interested in detecting SNPs within expressed mRNAs or the presence of gene fusions or alternative transcripts, it would be beneficial for all transcripts to be present equally within the library rather than their presence be waited by expression level. Accordingly, it would be useful to normalize the level of all transcripts to each other. Normalization of all fragments in a library is achieved by extension of Example 6 by using Cas9/guide RNA complexes specific to each fragment in the library.

Notwithstanding the appended claims, the disclosure is also defined by the following clauses:
1. A method for normalizing two or more nucleic acid samples, the method comprising:

contacting each of the two or more nucleic acid samples with a limiting amount of a normalization binding moiety that specifically binds to a common target in nucleic acids of each of the two or more nucleic acid samples to produce binding complexes in each of the two or more nucleic acid samples; and

separating the binding complexes from unbound nucleic acids in each of the two or more nucleic acid samples to normalize the two or more nucleic acid samples.
2. The method according to Clause 1, wherein the two or more nucleic acid samples comprises double stranded nucleic acids.
3. The method according to Clause 2, wherein the double stranded nucleic acids are dsDNAs.
4. The method according to Clause 3, wherein the two or more nucleic acid samples are next generation sequencing (NGS) libraries.
5. The method according to Clause 4, wherein the NGS libraries comprise double stranded nucleic acids comprising a common adapter.
6. The method according to Clause 7, wherein the common adapter comprises the common target.
7. The method according to any of the preceding clauses, wherein the common target comprises a target nucleic acid sequence and a normalization binding moiety comprises a sequence-specific normalization binding moiety.
8. The method according to Clause 7, wherein sequence-specific normalization binding moiety comprises a nucleic acid binding protein.
9. The method according to Clause 8, wherein the nucleic acid binding protein comprises a nuclease.
10. The method according to Clause 9, wherein the nuclease comprises a catalytically inactive nuclease.
11. The method according to Clause 10, wherein the nuclease comprises a nucleic acid guided DNA endonuclease.
12. The method according to Clause 11, wherein nucleic acid guided DNA endonuclease comprises a Cas9 nuclease.
13. The method according to Clause 10, wherein the nuclease comprises a restriction endonuclease.
14. The method according to Clause 8, wherein the nucleic acid binding protein comprises a TAL effector domain.
15. The method according to Clause 8, wherein the nucleic acid binding protein comprises zinc-finger protein (ZFP).
16. The method according to Clause 8, wherein the nucleic acid binding protein comprises a transcription factor.
17. The method according to any of Clauses 1 to 5, wherein the common target comprises a terminal motif and the normalization binding moiety comprises terminal motif binding protein.
18. The method according to any of Clauses 1 to 5, wherein the common target comprises a non-nucleic acid tag and the normalization binding moiety specifically binds to the non-nucleic acid tag.
19. The method according to any of the preceding clauses, wherein the normalization binding moiety comprises a purification domain.
20. The method according to Clause 19, wherein the purification domain comprises a his-tag, flag-tag, biotin, streptavidin, sumo tag, and any combination thereof.
21. The method according to Clause 20, wherein the separating comprises capturing the complexes via the purification domain.
22. The method according to Clause 21, wherein the purifying comprises use of a resin, a membrane, an affinity purification component, magnetic beads, or any combination thereof.
23. The method according to any of the preceding clauses, further comprising sequencing the normalized nucleic acid samples.
24. The method according to Clause 23, wherein the sequencing results in similar numbers of reads generated for each library.
25. The method according to any of the preceding clauses, wherein the method is performed in a single container.
26. The method according to Clause 25, wherein the container is selected from the group consisting of a tube, a plate, a multi-well array, and a droplet, or any combination thereof.
27. The method according to Clause 26, wherein the method is performed in a well of a multi-well array.
28. The method according to any of the preceding clauses, wherein the two or more nucleic acid samples are prepared from a cellular sample.
29. The method according to Clause 28, wherein the cellular sample comprises 10 cells or less.
30. The method according to Clauses 29, wherein the cellular sample comprises a single cell.
31. A kit for use in normalizing nucleic acid samples, the kit comprising:

a normalization binding moiety that specifically binds to a common target in each of two or more nucleic acid samples; and

a container for the normalization binding moiety.
32. The kit according to Clause 31, wherein the common target comprises a target nucleic acid sequence and the normalization binding moiety comprises sequence-specific normalization binding moiety.
33. The kit according to Clause 32, wherein sequence-specific normalization binding moiety comprises a nucleic acid binding protein.
34. The kit according to Clause 33, wherein the nucleic acid binding protein comprises a nuclease.
35. The kit according to Clause 34, wherein the nuclease comprises a catalytically inactive nuclease.
36. The kit according to Clause 35, wherein the nuclease comprises a nucleic acid guided DNA endonuclease.
37. The kit according to Clause 36, wherein nucleic acid guided DNA endonuclease comprises a Cas9 nuclease.
38. The kit according to Clause 35, wherein the nuclease comprises a restriction endonuclease.
39. The kit according to Clause 33, wherein the nucleic acid binding protein comprises a TAL effector domain.
40. The kit according to Clause 33, wherein the nucleic acid binding protein comprises zinc-finger protein (ZFP).
41. The kit according to Clause 33, wherein the nucleic acid binding protein comprises a transcription factor.
42. The kit according to Clause 31, wherein the common target comprises a terminal motif and the normalization binding moiety comprises terminal motif binding protein.
43. The kit according to Clause 31, wherein the common target comprises a non-nucleic acid tag and the normalization binding moiety specifically binds to the non-nucleic acid tag.
44. The kit according to any of Clauses 31 to 43, wherein the normalization binding moiety comprises a purification domain.
45. The kit according to Clause 44, wherein the purification domain comprises a his-tag, flag-tag, biotin, streptavidin, sumo tag, and any combination thereof.
46. The kit according to any of the preceding clauses, wherein the kit further comprises a separation component.
47. The kit according to Clause 46, wherein the separation component comprises a resin, a membrane, an affinity purification component, magnetic beads, or any combination thereof.
48. The kit according to any of Clauses 31 to 47, wherein the kit further comprises one or more NGS library preparation reagents.
49. The kit according to Clause 48, wherein the NGS library preparation reagents comprise a primer.
50. The kit according to Clause 50, wherein the primer is tagged.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method for normalizing two or more nucleic acid samples, the method comprising:
contacting each of the two or more nucleic acid samples with a limiting amount of a normalization binding moiety that specifically binds to a common target in nucleic acids of each of the two or more nucleic acid samples to produce binding complexes in each of the two or more nucleic acid samples; and
separating the binding complexes from unbound nucleic acids in each of the two or more nucleic acid samples to normalize the two or more nucleic acid samples.

2. The method according to claim 1, wherein the two or more nucleic acid samples comprises double stranded nucleic acids, preferably dsDNAs.

3. The method according to any of the preceding claims, wherein the two or more nucleic acid samples are next generation sequencing (NGS) libraries.

4. The method according to claim 3, wherein the NGS libraries comprise double stranded nucleic acids comprising a common adapter.

5. The method according to claim 4, wherein the common adapter comprises the common target.

6. The method according to any of the preceding claims, wherein the common target comprises a target nucleic acid sequence and a normalization binding moiety comprises a sequence-specific normalization binding moiety.

7. The method according to claim 6, wherein sequence-specific normalization binding moiety comprises a nucleic acid binding protein.

8. The method according to claim 7, wherein the nucleic acid binding protein comprises a nuclease, preferably a catalytically inactive nuclease.

9. The method according to claim 8, wherein the nuclease comprises a nucleic acid guided DNA endonuclease, preferably a Cas9 nuclease.

10. The method according to claim 7, wherein the nucleic acid binding protein comprises a TAL effector domain, a zinc-finger protein (ZFP) or a transcription factor.

11. The method according to any of claims 1 to 5, wherein the common target comprises a non-nucleic acid tag and the normalization binding moiety specifically binds to the non-nucleic acid tag.

12. The method according to any of the preceding claims, wherein the normalization binding moiety comprises a purification domain.

13. The method according to any of the preceding claims, further comprising sequencing the normalized nucleic acid samples.

14. The method according to any of the preceding claims, wherein the two or more nucleic acid samples are prepared from a cellular sample, preferably a single cell.

15. A kit for use in normalizing nucleic acid samples, the kit comprising:
a normalization binding moiety that specifically binds to a common target in each of two or more nucleic acid samples; and
a container for the normalization binding moiety.


SEQUENCE LISTING (12 sequences)

SEQ ID NO: 1 - DNA, 20 bp, Artificial sequence
  Synthetic sequence
aatgatacggcgaccaccga

SEQ ID NO: 2 - DNA, 24 bp, Artificial sequence
  Synthetic sequence
caagcagaagacggcatacgagat

SEQ ID NO: 3 - DNA, 33 bp, Artificial sequence
  Synthetic sequence
acactctttccctacacgacgctcttccgatct

SEQ ID NO: 4 - DNA, 34 bp, Artificial sequence
  Synthetic sequence
gtgactggagttcagacgtgtgctcttccgatct

SEQ ID NO: 5 - DNA, 30 bp, Artificial sequence
  Synthetic sequence
ccatctcatccctgcgtgtctccgactcag

SEQ ID NO: 6 - DNA, 23 bp, Artificial sequence
  Synthetic sequence
cctctctatgggcagtcggtgat

SEQ ID NO: 7 - protein, 8 aa, Artificial sequence
  Synthetic sequence
DYKDDDDK

SEQ ID NO: 8 - protein, 9 aa, Artificial sequence
  Synthetic sequence
YPYDVPDYA

SEQ ID NO: 9 - protein, 6 aa, Artificial sequence
  Synthetic sequence
HHHHHH

SEQ ID NO: 10 - protein, 10 aa, Artificial sequence
  Synthetic sequence
EQKLISEEDL

SEQ ID NO: 11 - protein, 8 aa, Artificial sequence
  Synthetic sequence
WSAPQFEK

SEQ ID NO: 12 - protein, 15 aa, Artificial sequence
  Synthetic sequence
GLNDIFEAQKIEWHE